(19) United States
(12) Patent Application Publication
(10) Pub. No.: US 2009/0275984 A1
(43) Pub. Date: Nov. 5, 2009

(54) REFORMING DEVICE

(75) Inventors: Gabriel Min Kim, West Friendship, MD (US); Benjamin Hyoungsol Lee, Waldwick, NJ (US); Scott Decker, Midlothian, VA (US); Rob Rasmussen, Pittsburgh, PA (US); Mark Kim, SE Mill Creek, WA (US); Shanna Renae Sprinkle, Oklahoma City, OK (US); Charles Chung-Jen Wang, Fremont, CA (US); Yuanting Zha, Monterey, CA (US); Aaron J. Noparstak, Northbrook, IL (US); In Hwa Lee, Baltimore, MD (US)

Correspondence Address: LEXYOUME IP GROUP, LLC, 5180 PARKSTONE DRIVE, SUITE 175, CHANTILLY, VA, 20151, US

(73) Assignee: Gabriel Min Kim, West Friendship, MD (US)

(21) Appl. No.: 12/434,831

(22) Filed: May 4, 2009

Related U.S. Application Data
(60) Provisional application No. 61/126,257, filed on May 2, 2008.

(30) Foreign Application Priority Data
Apr. 16, 2009 (KR) 10 2009 0033303

(51) Int. Cl. A61B 17/70

(52) U.S. Cl. 606/258; 606/259; 606/63

(57) ABSTRACT

The present invention relates to a reforming device. A reforming device according to the present invention includes a main body that is locked to bones of a human body and of which a length is extensible depending on growth of the human body; an external controller that transmits a wireless control signal for a length of the main body to be extended inside of the human body; and an internal controller that is mounted inside of the human body and extends the length of the main body depending on the wireless control signal.

### CROSS-REFERENCE TO RELATED APPLICATION

This application claims priority to and the benefit of U.S. Application No. 61/126,257 filed in the United States Patent and Trademark Office on May 2, 2008 and Korean Patent Application No. 10-2009-0033303 filed in the Korean Intellectual Property Office on Apr. 16, 2009, the entire contents of which are incorporated herein by reference.

### BACKGROUND OF THE INVENTION

(a) Field of the Invention The present invention relates to a reforming device.

(b) Description of the Related Art

Scoliosis is an illness having a symptom of a two-dimensional or three-dimensional curvature of a spine. A treatment for juvenile patients with early onset scoliosis (EOS) includes a method of reforming the curvature of the spine by locking a length-adjustable reforming device to both ends of the spine.

Further, the use of the reforming device is applicable in the case of asymmetries in limb lengths (i.e. a discrepancy between a patient's right and left arm lengths or alternatively a difference between their right and left leg lengths) due to a long-bone deformity, a bone fracture, the extension of a bone for cosmetic reasons, or other causes. In these situations it is possible to extend a length of the bone, reform the deformity, or treat the fracture by locking the reforming device to the bone.

As patients grow up, the length of the reforming device needs to be extended periodically. With the current technology, when the extension is needed, it requires an invasive operation necessitating a 5 to 6 inch incision into the patient's body to access the device during that particular surgery. Such an operation is performed once or twice a year. Such an invasive incision has a large infection risk, and the operation and recovery require a significantly amount of time and expense. Moreover, the concomitant illnesses of this patient population means the probability of them experiencing a complication is higher than the probability a healthy person might experience the same complication, therefore the operation is attended with a larger risk. The motivation for designing this reforming device stems is to eliminate the invasiveness of the lengthening procedure and it's attendant risks and to reduce cost

The above information disclosed in this Background section is only for enhancement of understanding of the background of the invention and therefore it may contain information that does not form the prior art that is already known in this country to a person of ordinary skill in the art.

### SUMMARY OF THE INVENTION

The present invention has been made in an effort to provide a reforming device having the advantages of reforming a bone more accurately while minimizing the need for an invasive operation.

An exemplary embodiment of the present invention provides a reforming device that includes a main body that is locked to bones of a human body and of which a length is extensible depending on growth of the human body; an external controller that transmits a wireless control signal for a length of the main body to be extended outside of the human body; and an internal controller that is mounted inside of the human body and extends the length of the main body.

The main body may include a rod; a rotating member that houses a part of the rod and is screw-joined to the rod; and a motor that rotates the rotating member to advance the rod to the outside of the rotating member.

Threads that engage with each other may be formed in the rod and the rotating members.

The thread of the rod and the thread of the rotating member may be self-locked to each other and do not move when the motor does not operate.

The main body further may include an internal case that is connected to the rotating member and the motor and transmits a rotational force from the motor to the rotating member.

The main body may further include a first case that houses the motor; and a second case that houses parts of the internal case, the rotating member, and the rod.

The main body may further include a support member that is mounted on the internal case.

The second case may include a wall formed inside thereof perpendicular to a longitudinal direction thereof and further include a thrust bearing mounted on the internal case, and the thrust bearing may be in contact with the wall.

A plurality of recess portions may be formed in the rod, and the main body may include a cap that closes a space between the second case and the rod and may include a plurality of convex portions corresponding to the recess portions.

The external controller may include a key pad through which the extension length of the main body is inputted; a display that displays the extension length inputted through the key pad; a first microcontroller that converts the extension length into the wireless control signal; and a first transmitter that transmits the wireless control signal from the first microcontroller to the internal controller.

The internal controller may include a first receiver that receives the wireless control signal; and a second microcontroller that processes a reception signal from the first receiver and outputs an output control signal for extending the length of the main body.

The internal controller may further include a second transmitter that transmits an actually extended length of the main body to the external controller.

The internal controller may further include a switch that switches on or off a power supply and the switch is controlled by an external magnetic field.

The external controller may further include a second receiver that receives the actually extended length of the main body and transmits the actually extended length to the first microcontroller, and the display displays the actually extended length.

The internal controller may further include a driver that supplies power to the main body in accordance with the output control signal.

The driver may include a first coil and may further include a driving controller that includes a second coil which induces a magnetic field to the first coil.

The reforming device may further include a sensor that measures an extension length of the rod.

The reforming device may further include a sensor that measures the rotation number of the motor.

Another embodiment of the present invention provides a reforming device locked to bones of a human body, which includes a rod; a rotating member that is screw-joined to the rod while housing a part of the rod; and a motor that operates in accordance with a wireless control signal from the outside to rotate the rotating member and advance a portion of the rod that is housed by the rotating member to the outside of the rotating member.

The reforming device may further include an internal case that is connected with the rotating member and the motor and transmits a rotational force from the motor to the rotating member; a first case that houses the motor; and a second case that houses parts of the internal case, the rotating member, and the rod.

The second case may include a wall formed inside thereof perpendicular to a longitudinal direction thereof and further include a thrust bearing mounted on the internal case, and the thrust bearing may be in contact with the wall.

According to the present invention, it is possible to reform bones more precisely in concordance with the growth of a patient while minimizing the number of operations and their attendant risks. Further, since a reforming device can be extended more frequently by the smaller unit than when the reforming device is manually extended, the reforming operation for the growth of the bones can be performed more precisely. Moreover, the length of the reforming device can be extended more precisely by readjusting the length depending on the actually extended length.

### BRIEF DESCRIPTION OF THE DRAWINGS

FIG. 1 is a schematic diagram of a reforming device according to an exemplary embodiment of the present invention.

FIG. 2 is a perspective view of a main body of a reforming device according to an exemplary embodiment of the present invention.

FIG. 3 is a cross-sectional view of a main body taken along line III-III of FIG. 2.

FIG. 4 is an exploded perspective view of a main body of a reforming device according to an exemplary embodiment of the present invention.

FIG. 5 is a cross-sectional view of a main body taken along line V-V of FIG. 4.

FIG. 6 is a cross-sectional view of a main body of a reforming device according to an exemplary embodiment of the present invention.

FIG. 7 is a block diagram of an internal controller of a reforming device according to an exemplary embodiment of the present invention.

FIG. 8 is a block diagram of an external controller of a reforming device according to an exemplary embodiment of the present invention.

FIG. 9 is a flow chart of a reforming method according to an exemplary embodiment of the present invention.

### DETAILED DESCRIPTION OF THE EMBODIMENTS

In the following detailed description, only certain exemplary embodiments of the present invention have been shown and described, simply by way of illustration. As those skilled in the art would realize, the described embodiments may be modified in various different ways, all without departing from the spirit or scope of the present invention. Accordingly, the drawings and description are to be regarded as illustrative in nature and not restrictive. Like reference numerals designate like elements throughout the specification.

Throughout the specification, in addition, unless explicitly described to the contrary, the word “comprise” and variations such as “comprises” or “comprising”, will be understood to imply the inclusion of stated elements but not the exclusion of any other elements. In addition, the terms “-er”, “-or” and “module” described in the specification mean units for processing at least one function and operation and can be implemented by hardware components or software components and combinations thereof.

Hereinafter, a reforming device according to an exemplary embodiment of the present invention will be described in detail with reference to the accompanying drawings.

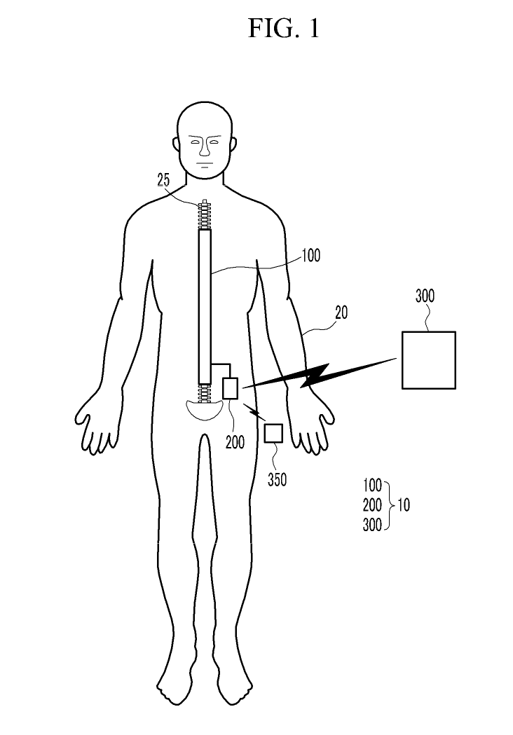

FIG. 1 is a schematic diagram of a reforming device according to an exemplary embodiment of the present invention.

Referring to FIG. 1, the reforming device 10 according to an exemplary embodiment of the present invention includes a main body 100, an internal controller 200, an external controller 300, and a driving controller 350.

The main body 100 is a part that is locked to a spine bone 25, a leg bone, an arm bone, etc. inside of a human body 20 to reform the bones. The length of the main body 100 is extended depending on control signals from the internal controller 200 and the external controller 300. That is, the main body 100 is transformed to suit growth or necessary correction of the human body 20.

The internal controller 200 is directly connected to the main body 100 to supply power to the main body 100, thereby extending the length of the main body 100. The internal controller 200 is mounted inside of the human body 20. For example, the internal controller 200 may be mounted subfascialy to the latissimus dorsi.

The external controller 300 transmits a wireless control signal from outside the human body 20 to the internal controller 200 inside of the human body 20 in accordance with a user's request to extend the length of the main body 100 if necessary.

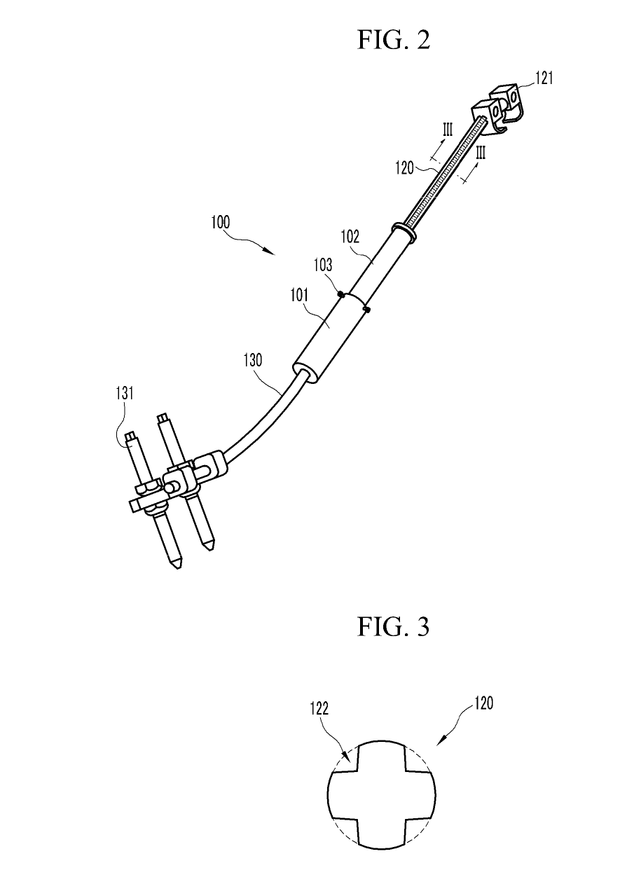

Hereinafter, referring to FIGS. 2 to 6, the main body of the reforming device according to an exemplary embodiment of the present invention will be described in detail.

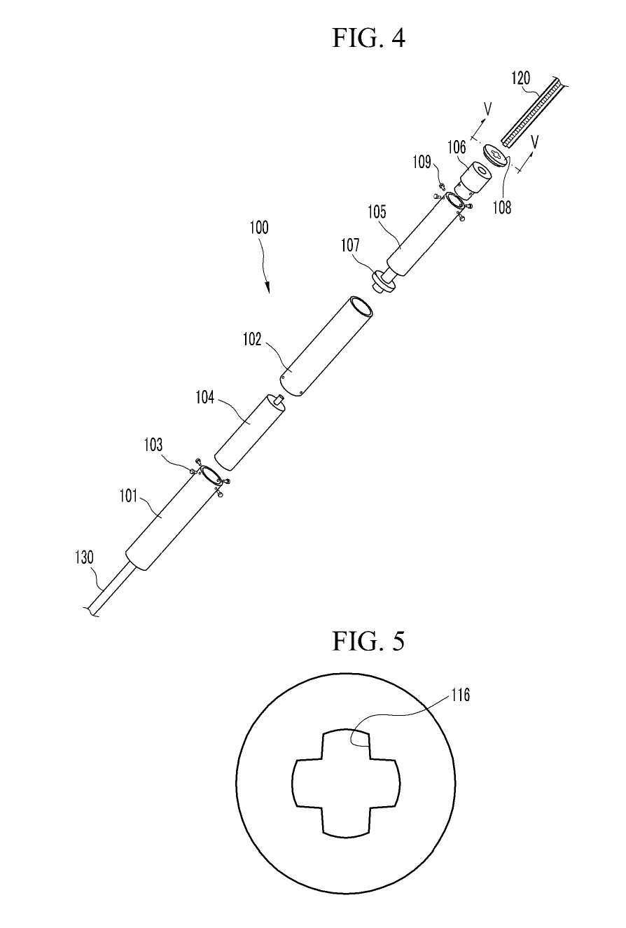

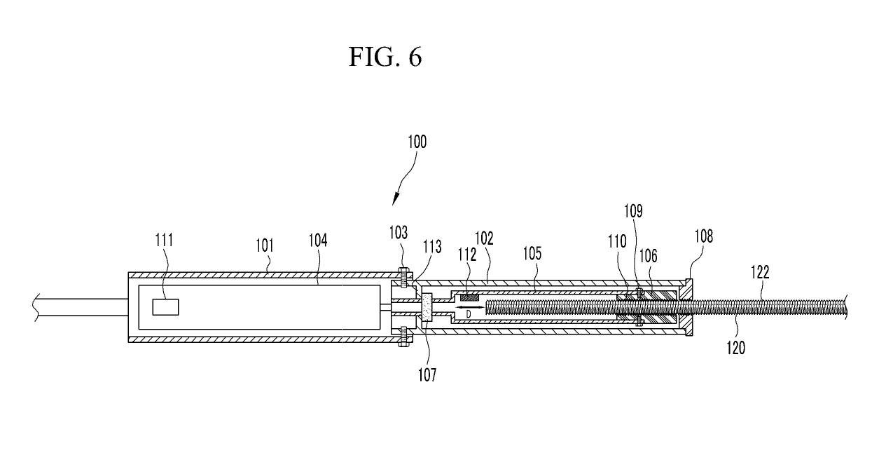

FIG. 2 is a perspective view of a main body of a reforming device according to an exemplary embodiment of the present invention. FIG. 3 is a cross-sectional view of a main body taken along line III-III of FIG. 2. FIG. 4 is an exploded perspective view of a main body of a reforming device according to an exemplary embodiment of the present invention. FIG. 5 is a cross-sectional view of a main body taken along line V-V of FIG. 4. FIG. 6 is a cross-sectional view of a main body of a reforming device according to an exemplary embodiment of the present invention.

First, referring to FIG. 2, the main body 100 of the reforming device according to an exemplary embodiment of the present invention includes a lower case 101, an upper case 102, a motor 104, an internal case 105, a rotating member 106, a support member 107, locking members 103 and 109, a cap 108, an upper rod 120, a lower rod 130, connection members 121 and 131, and sensors 111 and 112.

The lower case 101 houses the motor 104 and is locked and joined to the upper case 102 by the locking member 103. Connection of the lower case 101 and the upper case 102 is not limited to the locking member 103, but the lower case 101 and the upper case 102 may be screw-joined to each other.

The upper case 102 houses parts of the internal case 105, the rotating member 106, and the upper rod 120. A wall 113 which is perpendicular to a longitudinal direction of the upper case 102 is formed inside of the upper case 102.

The motor 104 is connected to the internal case 105. The motor 104 rotates the internal case 105 while it rotates.

The internal case 105 is locked and joined to the rotating member 106 by the locking member 109. A part of the internal case 105 is connected to the motor 104 through a wall 113 of the upper case 102.

The rotating member 106 houses the part of the upper rod 120. A thread 110 is formed on an internal wall surface of the rotating member 106.

The support member 107 supports the internal case 105 and may be a thrust bearing of which a load acts in a shaft direction. The support member 107 is in contact with the wall 113 of the upper case 102, such that pressure transmitted from the thoracic spine of the human body 20 (for example) to the main body 100 is not transmitted to the motor 104 but is transmitted to the lumber spine below the main body 100.

The cap 108 closes a space between an upper hole of the upper case 102 and the upper rod 120.

A thread 122 is formed on the surface of the upper rod 120. The thread 122 engages and contacts with the thread 110 that is formed on the internal wall surface of the rotating member 106. Therefore, the upper rod 120 and the rotating member 106 are screw-joined to each other. When the motor 104 operates, a length of the upper rod 120 screw-joined to the rotating member 106, which is exposed to the outside of the upper case 102 is extended while the internal case 105 and the rotating member 106 rotate. The threads 110 and 122 are self-locked to each other, such that the upper rod 120 does not rotate in an undesired direction when the motor 104 does not operate.

The lower rod 130 is connected to the lower case 101.

The connection members 121 and 131 connect and lock the upper rod 120 and the lower rod 130 to both sides of the spine 25, respectively. In the case when the main body 100 is used for not the spine 25 but other bones, the connection member 121 may be omitted.

Meanwhile, a plurality of recess portions 122 are formed in the upper rod 120 in a longitudinal direction of the upper rod. A plurality of convex portions 116 are formed in the cap 108 to correspond to the recess portions 122. The recess portions 122 of the upper rod 120 are joined to the convex portions 116 of the cap 108, such that the upper rod 120 is locked to the upper case 102 through the cap 108. Therefore, when the connection member 121 is not provided, the upper rod 120 is prevented from rotating.

When the sensor 111 is mounted on the motor 104, the rotation number of the motor 104 is measured and transmitted to the internal controller 200.

The sensor 112 is mounted in the internal case 105. A distance D between the upper rod 120 and a reference point is measured and transmitted to the internal controller 200.

Meanwhile, a thread may be formed on an internal wall surface of the internal case 105. In this case, the rotating member 106 is omitted and the upper rod 120 may be screw-joined to the internal case 105.

Meanwhile, in an exemplary of the present invention, a rotational force of the motor 104 is transmitted to the upper rod 120, such that the upper rod 120 advances to the outside of the upper case 102, but is not limited thereto and a linear actuator is provided instead of the motor 104, such that the upper rod 120 may advance to the outside of the upper case 102.

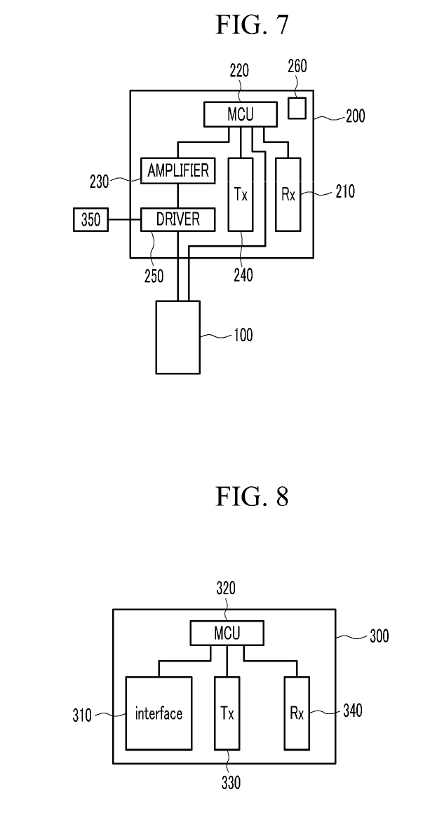

Hereinafter, the internal controller of the reforming device according to an exemplary embodiment of the present invention will be described in detail with reference to FIG. 7.

FIG. 7 is a block diagram of an internal controller and a driving controller of a reforming device according to an exemplary embodiment of the present invention.

Referring to FIG. 7, the internal controller 200 of the reforming device according to an exemplary embodiment of the present invention includes a receiver 210, a microcontroller 220, an amplifier 230, a transmitter 240, a driver 250, and a switch 260.

The receiver 210 receives the wireless control signal from the external controller 300 and serial-converts the wireless control signal to transmit the serial-converted signal to the microcontroller 220. Herein, the wireless control signal is information on the extension length of the upper rod 120 of the main body 100.

The microcontroller 220 analyzes the control signal serial-converted by the receiver 210 to calculate the extension length of the upper rod 120 and transmits the calculation result to the amplifier 230 as an output control signal. Further, the microcontroller 220 receives the information on the rotation number of the motor 104 from the sensor 111 by wire or wireless to determine how long the upper rod 120 is extended on the basis of the information and transmits the determination result to the transmitter 240. Further, the microcontroller 220 receives information on the distance D between the upper rod 120 and the reference point from the sensor 112 by wire line or wireless before and after the motor operates to determine the extension length of the upper rod 120 on the basis of the information and transmits the determination result to the transmitter 240.

The amplifier 230 amplifies and transmits the output control signal from the microcontroller 220 to the driver 250, and may be constituted by a transistor.

The transmitter 240 receives the information on the extension length of the upper rod 120 from the microcontroller 220 and transmits the information to the external controller 300.

The driver 250 operates the motor 104 of the main body 100 in accordance with the output control signal amplified by the amplifier 230. The driver 250 may be a transfer unit in accordance with a transcutaneous energy transfer system (TETS). In this case, the driver 250 may include rechargeable batteries such as a lithium ion battery and a coil. Further, the driver 250 may be a primary battery such as a lithium polymer battery.

The switch 260 switches on or off the internal controller 200 by a control outside of the human body 20. The switch 260 may operate by a magnetic field formed outside of the human body 20. The internal controller 200 is not always switched on, but the internal controller 200 is switched on only if necessary, that is, only when the length of the main body 100 is extended, such that the power of the internal controller 200 may be saved.

Hereinafter, the external controller 300 of the reforming device according to an exemplary embodiment of the present invention will be described in detail with reference to FIG. 8.

FIG. 8 is a block diagram of an external controller of a reforming device according to an exemplary embodiment of the present invention.

Referring to FIG. 8, the external controller 300 of the reforming device according to an exemplary embodiment of the present invention includes an interface 310, a microcontroller 320, a transmitter 330, and a receiver 340.

The interface 310 includes a key pad that receives information requested by the user, that is, the extension length of the upper rod 120 and a display that visually displays the extension length inputted through the key pad and an extension result to the user.

The microcontroller 320 converts the extension length inputted through the key pad into a control signal and transmits the control signal to the transmitter 330. The microcontroller 320 may be programmed to allow the extension length to be inputted from 0.1 mm to 2 mm by an interval of 0.1 mm. Further, the microcontroller 320 analyzes the extension result of the upper rod 120 from the internal controller 200 and displays the extension result through the display. Therefore, the user can verify an actually extended length in real time during an extension process.

The transmitter 330 receives the control signal from the microcontroller 320 and transmits the control signal to the internal controller 200.

The receiver 340 receives the extension result of the upper rod 120 from the internal controller 200 and transmits the extension result to the microcontroller 320.

The driving controller 350, as a part that allows the driver 250 of the internal controller 200 to be powered, may be the transfer unit in accordance with the transcutaneous energy transfer system (TETS). In this case, the driving controller 350 may include a coil. When current flows through the coil of the driving controller 350 to generate a magnetic field, the driving controller's magnetic field induces a magnetic field in a coil included in the internal controller 200 thereby generating a current in that coil and producing power for operating the motor 104. The power may directly operate the motor 104 or charge the rechargeable battery included in the internal controller 200. Therefore, when the lifespan of a driving source inserted into the human body 20 comes to an end, the motor 104 can continue to be driven without an operation to replace the driving source. If the driver 250 of the internal controller 200 includes the primary battery, the driving controller 350 may be omitted.

In the exemplary embodiment, the driving controller 350 is provided independent from the internal controller 200 and the external controller 300, but is not limited thereto and the driving controller 350 may be included in the external controller 300.

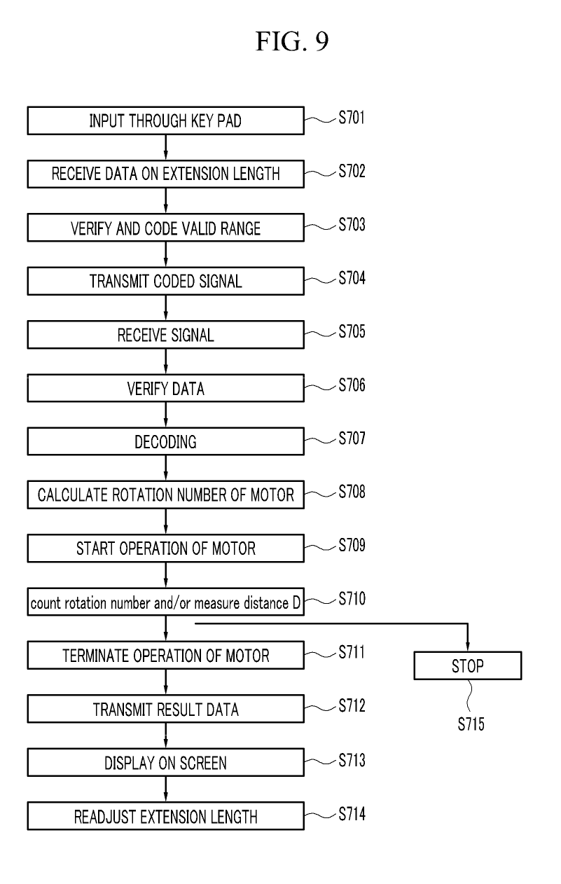

Hereinafter, a reforming method according to an exemplary embodiment of the present invention will be described in detail with reference to FIG. 9.

FIG. 9 is a flow chart of a driving method of a reforming device according to an exemplary embodiment of the present invention.

Referring to FIG. 9, first, a user inputs an extension length of an upper rod 120 of a reforming device through a key pad of an external controller 300 as an extension is necessary (S701).

Therefore, a microcontroller 320 of the external controller 300 receives data on the extension length (S702). Subsequently, the microcontroller 320 verifies a valid range for the extension length in the received data and codes the data (S703).

Thereafter, the coded data is transmitted to an internal controller 200 as a wireless control signal through a transmitter 330 (S704).

A receiver 210 of the internal controller 200 receives the coded data (S705).

Thereafter, the microcontroller 200 verifies the data (S706). The data verifying process is a process of determining whether or not the wireless control signal received by the internal controller 200 is a signal for the reforming device among numerous wireless signals that exist in an external environment. The microcontroller 220 decodes the verified data (S707) and calculates the rotation number of a motor 104 (S708). Subsequently, the microcontroller 220 starts an operation of the motor 104 (S709). Therefore, a sensor 111 of the motor 104 counts the rotation number and/or sensor 112 measures the distance D between the upper rod 120 and a reference point (S710). When the microcontroller 220 receives information on the rotation number and/or distance D and the motor 104 rotates at the same rotation number as the calculated rotation number and/or motor 104 performs enough rotations to extend the upper rod 120 by user's specified extension length, the microcontroller 220 terminates the operation of the motor 104 (S711). Thereafter, a transmitter 240 of the internal controller 200 transmits result data—which is information on the actual extended length of the upper rod 120 of the main body 100 which is calculated from the rotation number of the motor 104 and/or distance D between the upper rod 120 and a reference point measured by 112—to a receiver 340 of the external controller 300 (S712).

The transmitted result data are displayed through a display (S713). Therefore, the user can recognize the result.

Thereafter, the user checks the result data and determines whether or not the extension length needs to be readjusted. When the user determines that the extension length needs to be readjusted, the user readjusts the extension length (S714). That is, the result data transmitting step (S712) is repeated from the above-mentioned valid range verifying step (S703).

Meanwhile, after the operation of the motor is started (S709), the operation of the motor can be stopped by a user's judgment (S715) before the operation of the motor is terminated (S711).

According to the present invention, except for when the main body 100 of the reforming device is mounted on and removed from the human body 20, the length of the reforming device can be extended depending on growth of a bone by an external manipulation without an additional operation. Further, since the reforming device can be extended more frequently by smaller units than when the reforming device is manually extended, the reforming operation for the growth of the bone can be performed more precisely. Moreover, the length of the reforming device can be extended more precisely by readjusting the length depending on the actual extended length.

The above-mentioned exemplary embodiment of the present invention contains designs for a device and a method that do not need to be implemented fully and simultaneously, but may be implemented by a program that can realize functions corresponding to components of the exemplary embodiment of the present invention or a recording medium in which the program is recorded.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

What is claimed is:

1. A reforming device, comprising:
a main body that is locked to bones of a human body and of which a length is extensible depending on induced and/or natural growth of the human body;
an external controller outside of the human body that transmits a wireless control signal for a length of the main body to be extended; and
an internal controller that is mounted inside of the human body and extends the length of the main body depending on the wireless control signal.

2. The reforming device of claim 1, wherein:
the main body comprises,
a rod,
a rotating member that houses a part of the rod and is screw-joined to the rod; and
a motor that rotates the rotating member to advance the rod to the outside of the rotating member.

3. The reforming device of claim 2, wherein:
threads that engage with each other are formed in the rod and the rotating members, respectively.

4. The reforming device of claim 3, wherein:
the thread of the rod and the thread of the rotating member are self-locked to each other and do not move when the motor does not operate.

5. The reforming device of claim 2, wherein:
the main body further comprises,
an internal case that is connected to the rotating member and the motor and transmits a rotational force from the motor to the rotating member and houses a part of the rod.

6. The reforming device of claim 5, wherein:
the main body further comprises,
a first case that houses the motor; and
a second case that houses the internal case, the rotating member, and at least a part of the rod.

7. The reforming device of claim 6, further comprising:
a support member that is mounted on the internal case.

8. The reforming device of claim 7, wherein:
the second case comprises a wall formed inside thereof perpendicular to a longitudinal direction thereof,
the support member comprises a thrust bearing, and
the thrust bearing is in contact with the wall.

9. The reforming device of claim 6, wherein:
a plurality of recess portions is formed in the rod, and
the main body comprises a cap that closes a space between the second case and the rod and the cap comprises a plurality of convex portions corresponding to the recess portions.

10. The reforming device of claim 1, wherein:
the external controller comprises,
a key pad through which the extension length of the main body is inputted;
a display that displays the extension length inputted through the key pad;
a first microcontroller that converts the extension length into the wireless control signal; and
a first transmitter that transmits the wireless control signal from the first microcontroller to the internal controller.

11. The reforming device of claim 10, wherein:
the internal controller comprises,
a first receiver that receives the wireless control signal; and
a second microcontroller that processes the wireless control signal from the first receiver and outputs an output control signal for extending the length of the main body.

12. The reforming device of claim 11, wherein:
the internal controller further comprises a second transmitter that transmits an actually extended length of the main body to the external controller.

13. The reforming device of claim 12, wherein:
the internal controller further comprises a switch that switches on or off a power supply and the switch is controlled by an external magnetic field.

14. The reforming device of claim 12, wherein:
the external controller further comprises,
a second receiver that receives the actually extended length of the main body and transmits the actually extended length to the first microcontroller, and
the display displays the actually extended length.

15. The reforming device of claim 12, wherein:
the internal controller further comprises a driver that supplies power to the main body in accordance with the output control signal.

16. The reforming device of claim 15, wherein:
the driver comprises a first coil, and the reforming device further comprises a driving controller that comprises a second coil which induces a magnetic field to the first coil.

17. The reforming device of claim 2, further comprising:
a sensor that measures an extension length of the rod.

18. The reforming device of claim 2, further comprising:
a sensor that measures the rotation number of the motor.

19. A reforming device locked to bones of a human body, comprising:
a rod;
a rotating member that is screw-joined to the rod while housing at least a part of the rod; and
a motor that operates in accordance with a wireless control signal from the outside to rotate the rotating member and advance the rod to the outside of the rotating member.

20. The reforming device of claim 19, further comprising:
an internal case that is connected with the rotating member and the motor and transmits a rotational force from the motor to the rotating member;
a first case that houses the motor;
a second case that houses the internal case, the rotating member, and at least a part of the rod and comprises a wall formed inside thereof to be perpendicular to a longitudinal direction thereof; and
a thrust bearing that is mounted on the internal case,
wherein the thrust bearing is in contact with the wall.
